(19) United States
(12) Patent Application Publication
(10) Pub. No.: US 2020/0078471 A1
(43) Pub. Date: Mar. 12, 2020

(54) RENAL CLEARABLE DRUG DELIVERING NANOPARTICLES AND METHODS OF USE THEREFOR

(71) Applicant: Board of Regents, The University of Texas System, Austin, TX (US)

(72) Inventors: Jie ZHENG, Plano, TX (US); Chuanqi PENG, Richardson, TX (US)

(73) Assignee: Board of Regents, The University of Texas System, Austin, TX (US)

(21) Appl. No.: 16/552,359

(22) Filed: Aug. 27, 2019

Related U.S. Application Data
(60) Provisional application No. 62/723,444, filed on Aug. 27, 2018.

(51) Int. Cl. A61K 47/69; A61K 47/60; A61K 31/7105; C12N 15/113; A61K 49/18; A61P 35/00; B82Y 5/00; A61K 31/704

(52) U.S. Cl. CPC A61K 47/6929; A61K 47/6923; A61K 47/60; A61K 31/7105; C12N 15/113; C12N 2320/32; A61P 35/00; B82Y 5/00; A61K 31/704; C12N 2310/14; C12N 2310/351; A61K 49/1824

(57) ABSTRACT

The present disclosure relates to the design and use of specific drug-nanoparticle constructs where the loading of the drugs onto nanoparticle are through noncovalent interactions. The obtained nanoconstructs are smaller than 10 nm in hydrodynamic diameter in the physiological environment, are highly resistant to serum protein adsorption, can penetrate the tumor core deeply via passive diffusion, can be retained in the tumor cores through enhancement permeability and retention effect, can rapidly diffuse across interendothelial junctions but can also be rapidly eliminated from the background tissues and normal organs. In addition, “off-target” drug-particle nanoconstructs have very low accumulation in the liver and can be eliminated through the urinary system. Through the use of these nanoparticles, the toxicity and side effect of chemodrugs is significantly reduced and the therapeutic index greatly improved.

### PRIORITY CLAIM

This application claims benefit of priority to U.S. Provisional Application Ser. No. 62/723,444, filed Aug. 27, 2018, the entire contents of which are hereby incorporated by reference.

### STATEMENT REGARDING FEDERALLY SPONSORED RESEARCH OR DEVELOPMENT

This invention was made with government support under Grant No. R01 DK103363 awarded by the National Institutes of Health. The government has certain rights in the invention.

Other funding for the work described here comes from Grant No. RP160866 awarded by the Cancer Prevention Research Institute of Texas.

### BACKGROUND

### A. Field

The disclosure relates generally to the fields of medicine, diagnostics, therapy, and imaging. More particularly, it relates to the design, preparation and use of stable, renal clearable drug-delivering nanoparticle systems.

### B. Related Art

Engineered nanoparticles (NPs) have been widely used in the cancer-selective delivery of chemodrugs since the administrated nanoparticles can prevent the drugs from rapid liver uptake or kidney elimination; so that drug molecules can be retained in the blood stream for a longer period of time and more effectively accumulate in tumors through enhanced permeability and retention

(EPR) effect (Davis & Shin, 2008; Iyer et al., 2006). Guided by this fundamental delivery principle, a large number of nanoparticle-based delivery systems including recent cell-membrane based ones have been developed to prolong blood retention of chemodrugs for improved delivery (Luk & Zhang, 2015; Shi et al., 2017). However, clinical translation of cancer nanomedicines remains fairly challenging because in vivo interactions of engineered nanoparticles with normal and cancerous tissues raise new biological barriers for maximizing therapeutic efficacy and minimizing side effects of chemodrugs (Blanco et al., 2015). For example, enhancing blood retention of chemodrugs with engineered nanoparticles larger than 6 nm allows drug molecules to be confined more in the blood vessels and escape rapid kidney filtration (Soo Choi et al., 2007); however, it also slows down the elimination of “off-target” chemodrugs, which eventually end up in the liver and other vital organs and in turn increases long-term health risk (Tsoi et al., 2016). Increasing nanoparticle size above 50 nm for stronger EPR effect often reduces the extravasation of chemodrugs into the normal tissues but also limits the penetration of chemodrugs to the tumor cores due to the high interstitial fluid pressure and dense extracellular matrix in the tumor microenvironment (Jain & Stylianopoulos, 2010). Moreover, since macrophage uptake of engineered nanoparticles is strongly size-dependent, engineered nanoparticles are often taken up by macrophages in the tumor microenvironment, which significantly reduces their targeting to cancer cells (Dait et al., 2018). Although many new strategies such as targeting delivery, controlled release have been developed to tackle these challenges, at the fundamental level, all these strategies still rely on prolonging blood retention of chemodrugs to achieve more efficient tumor targeting. Thus, a fundamental question in the drug delivery is whether engineered nanoparticles can be used to rapidly and efficiently deliver chemodrugs without the need of prolonging blood retention of chemodrugs and without sacrificing chemodrug tumor penetration in the meantime the retention of “off-target” chemodrugs in normal tissues and vital organs can be significantly shortened?

### SUMMARY

In accordance with the present disclosure, there is provided a composition comprising a noble metal nanoparticle comprising at least a first drug, wherein the surface is derivatized with polyethylene glycol and benzyl mercaptan or derivative thereof, and wherein said drug is non-covalently associated with the benzyl mercaptan or derivative thereof. The noble metal may be gold. The nanoparticle may be about 0.5 nm to 10 nm in diameter, or about 1 nm to 5 nm in diameter. The nanoparticle core may be about 0.5 nm to 5 nm in diameter. The nanoparticle may be derivatized with benzyl mercaptan, such as with a benzyl mercaptan methoxy derivative, mercaptobenzoic acid, aminothiophenol, methylbenzenethiol, thiolated pyrene, thiolated nucleobases or other aromatic thiol. The nanoparticle may comprise a ligand capable of binding to at least one cellular component, such as a tumor marker. The ratio of polyethylene glycol and a secondary ligand may be in the range of 1:0.1, 1:0.25, 1:0.5, 1:1, 1:2, 1:4, 1:8. There may be five drug molecules loaded.

The polyethylene glycol and mercaptobenzoic acid shield loaded drugs and stabilize the nanoparticles. The nanoparticle may be less than 10 nm in diameter in a physiological environment. The nanoparticle may be renally clearable. The nanoparticle may be resistant to serum protein binding. The blood residence half-time of the nanoparticle after administration of the gold nanoparticle to a subject may range from about 2 hours to about 25 hours. The drug may be a protein, a nucleic acid (e.g., siRNA), or a small molecule, such as a chemotherapeutic (e.g., doxorubicin) or imaging agent. The chemotherapeutic may be an anthracycline drug, such as doxorubicin, daunorubicin, epirubicin, idarubicin. The chemotherapeutic may be a platinum-based chemotherapeutic (e.g., cisplatin, oxaliplatin, carboplatin, or nedaplatin). The imaging agent may be a near-infrared imaging agent, such as indocyanine green and methylene blue. The imaging agent may be a gadolinium complex. The nanoparticle may comprise a second drug. The nanoparticle may comprise an imaging probe or radiotracer. The noble metal may consist of, comprise, or consist essentially of silver, copper, platinum, or carbon, and optionally is luminescent. The size of the nanoparticle may be less than 3 nm. The non-covalent bond may involve pi-pi stacking or electrostatic interactions.

The blood residence half-time of the administrated nanoparticle may range from about 10 min to about 25 hours. The nanoparticle may achieve rapid and high drug accumulation in tumor tissues as compared to a nanoparticle lacking benzyl mercaptan derivatization. The nanoparticle may target tumor effectively based on the enhanced permeability and retention effect in tumor tissues and density dependent nanoparticle margination in blood vessels as compared to a nanoparticle lacking benzyl mercaptan derivatization. The nanoparticle may reach a penetration of about 40% or more of tumor cells within a distance of about 100 p.m of a blood vessel in the tumor environment. The nanoparticle may prolong the tumor retention of drugs, therefore have high local drug concentration in tumor for long time. The nanoparticle may show enhanced release in tumors compared to normal tissues due to the acidic pH value in tumor environment as compared to a nanoparticle lacking benzyl mercaptan derivatization. The nanoparticle may show enhanced release in tumors compared to normal tissues also due to the high glutathione concentration in tumor tissues as compared to a nanoparticle lacking benzyl mercaptan derivatization. The nanoparticle may target solid tumors, including primary and metastatic ones, and including breast cancer, which may have metastasized to the lung. The nanoparticle may penetrate normal tissues via endothelial junctions due to the small size. The nanoparticle may be smaller than interendothelial junctions and permeable into skeletal muscle (gastrocnemius) of a subject show reduced retention in normal organs and tissues, such as skeletal muscle, heart, lung, and liver as compared to a nanoparticle lacking benzyl mercaptan derivatization.

In another embodiment, there is provided a method of treating a subject having a hyperproliferative disease comprising administering to the subject a composition as defined above. The composition may be administered to said subject orally, intravenously, nasally, subcutaneously, intramuscularly or transdermally. The hyperproliferative disease may be a benign hyperproliferative disease. The hyperproliferative disease may be a malignancy. The subject may be a human subject, or a non-human mammalian subject. The malignancy may be recurrent, metastatic or multiple drug dependent.

The method may further comprise administering to a subject a second anti-cancer therapy, such as a distinct chemotherapy, radiotherapy, immunotherapy, toxin therapy, or surgery. The second cancer therapy may be a distinct drug therapy delivered on the same nanoparticle as said first drug or on a different nanoparticle as said first drug.

Also provided is a method for preparation of renal clearable drug delivering nanoparticles as described above.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description.

It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. In vivo transport of anticancer drug, Doxorubicin (DOX), with or without being loaded on renal clearable drug delivery system (RC-DDS, DOX@AuNPs) and non-renal clearable DDS (NRC-DDS, NRC-DOX@AuNPs). (FIG. 1A) The RC-DDS significantly enhanced the urinary clearance of “off-target” drugs while NRC-DDS and free DOX showed negligible or inefficient renal clearance. (FIG. 1B) In the liver, RC-DDS significantly reduced drug accumulation while NRC-DDS increased DOX accumulation and free DOX was rapidly metabolized. (FIG. 1C) RC-DDS accelerated the clearance of DOX from normal tissues as muscle, heart and lungs, etc., whereas, the unwanted DOX released from NRC-DDS retained in normal tissues for long time and induced systemic toxicity. (FIG. 1D) RC-DDS enhanced DOX delivery through EPR effect without sacrificing the deep penetration of DOX while NRC-DDS significantly reduced DOX tumor penetration.

FIGS. 2A-C. Design and characterization of the renal clearable DOX-AuNP constructs (DOX@AuNPs). (FIG. 2A) Scheme of the size and structure of renal clearable DOX@AuNPs. The delivery vector consists of the metal core (Au358, 2.06±0.18 nm in diameter, FIGS. 3A-E), surface coating with poly(ethylene glycol) thiol (PEG-SH, m.w. 800) and 4-mercaptobenzoic acid (MBA); the drug loading is achieved through the pi-pi (it-it) stacking interaction between DOX and MBA on AuNPs. (FIG. 2B) Hydrodynamic diameters (HDs) of DOX@AuNPs and AuNPs in phosphate-buffered saline (PBS) at room temperature. Loading DOX onto the AuNPs leads to 1.2-nm increase in HD. (FIG. 2C) UV-Vis absorption spectra of DOX@AuNPs, AuNPs and free DOX in aqueous solution. The loaded DOX exhibited 8-nm redshift (from 488 to 496 nm) compared with free DOX, indicating the formation of J-aggregates. Inset, characteristic absorption peaks of loaded DOX (plotted by absorbance subtraction of AuNPs from DOX@AuNPs) compared with free DOX.

FIGS. 3A-E. Quantification of surface ligands on renal clearable AuNP (Au₃₅sPEG₆₁MBA₉₁). (FIGS. 3A-B) Molecular structures and characteristic protons of 4-mercaptobenzoic acid (MBA) (FIG. 3A) and poly(ethylene glycol) methyl ether thiol (PEG-SH, m.w. 800, n˜16) (FIG. 3B). (FIG. 3C) Nuclear magnetic resonance (NMR) spectra of PEG-SH (1.00%, w/v), AuNPs (1.00%, w/v), and mixture of AuNPs and PEG-SH (0.50%, 0.50%, w,w/v) in chloroform-d (CDC13). (FIGS. 3D-E) Transmission electron microscopy image (FIG. 3D) and size distribution (FIG. 3E) of the AuNPs. The AuNPs showed the average core size of 2.06±0.18 nm. Scale bar, 5 nm.

For calculations:²⁵⁻²⁸ NMR signals for major protons: MBA: H 1, 7.26 ppm, singlet; H 2, 8.01 ppm, singlet (broadened on AuNPs);

PEG-SH: H 3, 3.38 ppm, singlet; H 4, 3.64 ppm, triplet; H 5, 3.55 ppm, triplet; H 6, 2.70 ppm, quartet.

(1) H 4: PEG-SH (1.00%, w/v) : PEG-SH (0.50%, w/v)+AuNPs (0.50%, w/v) : AuNPs (1.00%, w/v)=27.57 : 18.83 : 10.03=(13.78×2) : (13.78 +5.04) : (5.01×2)

PEG % on AuNPs=Signal from AuNPs/Signal from PEG-SH=10.03/27.57=36.4% (w/w)

(2) For AuNPs: Signal H 1 (2 protons): Signal H 3, 4, 5 (3 +16×4+2=69 protons)=21.36:0.92=69×0.309:2×0.459;

Therefore, NPEG: NMBA˜2:3;

MBA % on AuNPs=PEG % MMBA/MPEGXNMBANPEG=36.4×153/799×1.5=10.5% (w/w);

(3) For AuNPs, Au % on AuNPs=100%−PEG%−MBA%=53.1% (w/w).

(4) Structure of AuNPs:

The average diameter of gold core was 2.06±0.18 nm, therefore, the number of gold per particle, N_{Au}=(R_{AuNP}/R_{Au})³=((2.06/2)/0.145)³=358; 358×M_{Au}=358×197=70,526, which was 53.1%; Therefore, N_{PEG}=61; 61 ×M_{PEG}=61×799=48,346, which was 36.4%; N_{MBA}=91; 91×M_{MBA}=91×153=13,946, which was 10.5%. The structure of AuNPs was estimated as Au₃₅₈PEG₆₁MBA₉₁. The average particle weight was about 132,818. The structure of PEG-AuNPs (same core size) was estimated as Au₃₅₈PEG₁₂₆ by using the same method.

FIGS. 4A-D. Characterization of non-renal clearable DDS. (FIGS. 4A-B) TEM image (FIG. 4A) and size distribution (FIG. 4B) of the non-renal clearable PEG/MBA-AuNPs (NRC-AuNPs). The NRC-AuNPs after surface modification exhibited an average core size of 23.6±4.3 nm, comparable to the NRC-PEG-AuNPs. (FIG. 4C) HD of NRC-DOX@AuNPs (30.9±6.2 nm). (FIG. 4D) UV-Vis absorbance spectra of NRC-AuNPs and NRC-DOX@AuNPs (the loading capacity of 57,900±20,700 DOX/NP).

FIGS. 5A-D. Drug loading studies. (FIG. 5A) DOX loading of AuNPs with differently charged secondary ligands, such as like 4-mercaptobenzoic acid (MBA), 4-aminothiophenol (ATP), 2-phenylethanethiol (PET), which all showed high loading capacity. (FIG. 5B) The drug loading (DOX in this case) was achieved with only certain range of MBA-to-PEG surface ligand ratio with the high loading capacity and adequate physiological stability. For the dual-ligand AuNPs with MBA-to-PEG ratio lower than 4:1, the stable and efficient drug loading was achieved. The further increase of MBA-to-PEG induced the higher hydrophobicity of the AuNPs and instability of DOX-loaded AuNPs eventually. (FIG. 5C) Optimal drug loading capacities for different drug molecules, such as anticancer drugs (DOX, cisplatin, and oxaliplatin), clinically approved imaging agents (ICG), and siRNAs. (FIG. 5D) Purification of drug-loaded AuNPs from free drug molecules via size exclusion column, where DOX (red) and ICG (green) are the drug representatives for purification.

FIGS. 6A-C. Drug loading studies with different types of drugs. The loaded drugs are characterized with Ultraviolet-Visible-Near infrared absorbance for different types of drugs. (FIG. 6A) The representative anticancer drugs are included as anthracycline drugs as doxorubicin, DOX, platinum-based drugs as cisplatin, CPt, and oxaliplatin, OPt, which are the essential drugs in clinics; meanwhile, the AuNPs can also load and deliver the highly toxic drugs, such as mithramythin, MTH, which is the commercially available anticancer drugs with the most liver toxicity. Hence, the efficient delivery and renal clearance of this type of highly toxic drugs which greatly increase the efficacy as well as safety. (FIG. 6B) The representative molecules as imaging probes are included as fluorescent imaging probes as the clinically approved indocyanine green, ICG and methylene blue, MB, as well as the contrast agent gadolinium for magnetic resonance imaging (MRI). (FIG. 6C) The other type of drug molecules is the small interfering RNA.

FIGS. 7A-G. Physiological stability, clearance and biodistribution of the DOX@AuNPs. (FIG. 7A) HDs of DOX@AuNPs (5 μM) in PBS with and without the addition of human serum albumin (HSA, 5 μM) for 3 hr at 37° C. The unchanged HD and absence of larger nanoparticles indicated that the DOX@AuNPs had low affinity to serum protein binding. (FIG. 7B) Gold-based renal clearance of the injected DOX@AuNPs, dual-ligand AuNPs, PEG-AuNPs and non-renal clearable DOX@AuNPs (NRC-DOX@AuNPs) in 24 hr post-injection (p.i., n=3). Error bar indicates s.d. ****P<0.0001 (Student's t-test). (FIG. 7C) DOX-based renal clearance of the injected DOX@AuNPs, free DOX and NRC-DOX@AuNPs in 24 hr p.i. (n=3). The renally cleared DOX for the injected DOX@AuNPs was 3.6 and 6.5 times higher than that of the injected DOX, NRC-DOX@AuNPs, respectively, indicating the enhanced renal elimination of “off-target” drug. (FIG. 7D) DOX distribution in kidney and liver of the mice injected with DOX@AuNPs, NRC-DOX@AuNPs and free DOX at 1 and 24 hr p.i. (n=3). P<0.005, ****P<0.0001. (FIG. 7E) Pharmacokinetics of the injected DOX@AuNPs, NRC-DOX@AuNPs and free DOX within 24 hr p.i. (n=3). (FIG. 7F) Muscle DOX distribution for the injected DOX@AuNPs, NRC-DOX@AuNPs and free DOX at 1 and 24 hr p.i., where DOX@AuNPs showed more significant elimination from skeletal muscle than NRC-DOX@AuNPs and free DOX (n=3). *P<0.05; NS, not significant. (FIG. 7G) DOX distribution in vital organs as heart and lung of the mice injected with DOX@AuNPs, NRC-DOX@AuNPs and free DOX at 1 and 24 hr p.i. (n=3). *P<0.05, **P<0.01, ***P<0.005.

FIGS. 8A-B. Biodistribution and elimination of DOX@AuNPs and NRC-DOX@AuNPs. (FIG. 8A) Biodistribution of DOX@AuNPs and NRC-DOX@AuNPs based on gold concentrations at 24 hr p.i (n=3). The NRC-DOX@AuNPs showed sever accumulation in RES, including liver, spleen and lung, whereas the DOX@AuNPs showed significant lower distribution. (FIG. 8B) Photo image of the mice organs after successive treatment of DOX@AuNPs, NRC-DOX@AuNPs and PBS on 15 days p.i. Notably, the NRC-DOX@AuNPs showed massive accumulation and darkened the color of RES organs as lung, liver and spleen as well as kidney. The long-term exposure of NRC-DOX@AuNPs would be one major concern on the biosafety and limitation of the DDS.

FIGS. 9A-D. Toxicity studies of the renal clearable DOX@AuNPs compared to non-renal clearable DOX@AuNPs and free DOX. (FIG. 9A) Body weight changes of CD-1 mice after being successively treated with DOX@AuNPs, NRC-DOX@AuNPs, equivalent DOX and PBS control (n=4). Treatment of free DOX as well as NRC-DOX@AuNPs induced severe body weight loss (˜10%), mortality (25%) and related adverse effects in 10 days, whereas DOX@AuNPs treatment showed no significant difference in body weight compared to control group. P<0.005. (FIGS. 9B-D) Blood chemistry analysis of mice treated with DOX@AuNPs, NRC-DOX@AuNPs, free DOX and PBS on 15 day p.i.; alanine aminotransferase, ALT, and aspartate transaminase, AST (FIG. 9B), alkaline phosphatase, ALKP, and albumin (FIG. 9C), and blood urea nitrogen, BUN and serum creatinine, CREA (FIG. 9D) (n=3). Error bar indicates s.d., box indicates median and s.e.m. *P<0.05, **P<0.01, P<0.005, ****P<0.0005.

FIGS. 10A-B. Hepatotoxicity study of DOX@AuNPs and NRC-DOX@AuNPs. (FIG. 10A) H&E staining of liver tissues with successive treatment of DOX@AuNPs, NRC-DOX@AuNPs, equivalent free DOX and PBS on 15 days p.i. Scale bar, 50 μm. Anisocaryosis and anisocytosis of hepatocytosis²² was evident for the NRC-DOX@AuNPs and free DOX treatments, which was minimized with DOX@AuNPs treatment. (FIG. 10B) Analysis of the nuclei size for the hepatocytes with DOX@AuNPs, NRC-DOX@AuNPs free DOX and PBS treatments. The hepatocytes with free DOX treatment as well as NRC-DOX@AuNPs showed significant higher distribution width (due to the massive liver uptake and toxicity of genotoxic agent), which was absent in DOX@AuNPs and PBS treatment. This indicated the impairment to hepatocytes from free DOX and NRC-DOX@AuNPs, consistent with blood chemistry study. Box indicates median and 25-75% interquartile range. Error bar indicates s.d. DW, distribution width=100 (s.d./mean). In addition, no observable pathological signs in heart, kidney, spleen and muscle tissues for both DOX@AuNPs, NRC-DOX@AuNPs and free DOX, which might be due to the limited dose.

FIGS. 11A-D. Toxicity and tolerance of DOX@AuNPs, NRC-DOX@AuNPs and free DOX. (FIG. 11A) Body weight change of mice administrated with DOX@AuNPs (containing DOX 15 and 20 mg/kg), NRC-DOX@AuNPs (containing DOX 5, 10 and 15 mg/kg), DOX 10, 15 and 20 mg/kg, and PBS, respectively, in 10 days p.i. (n=4). (FIGS. 11B-D) Blood chemistry analysis for liver functions as alanine aminotransferase, ALT and aspartate transaminase, AST (FIG. 11B), alkaline phosphatase, ALKP and albumin (FIG. 11C) levels, and renal functions as blood urea nitrogen, BUN and creatinine, CREA (FIG. 11D) levels in serum for the mice with DOX@AuNPs (15 or 20 mg/kg), NRC-DOX@AuNPs (5, 10 and 15 mg/kg), DOX (10 or 15 mg/kg) and PBS treatment 10 days p.i. (n=3). *P<0.05. Combined with the body weight change and blood chemistry results, the maximal tolerance dose (MTD, in terms of DOX) for DOX@AuNPs, NRC-DOX@AuNPs and free DOX were 15, 10 and 10 mg/kg body weight.

FIGS. 12A-G. Targeting efficiency, tumor penetration of the renal clearable DOX@AuNPs and non-renal clearable DOX@AuNPs. (FIG. 12A) Pharmacokinetics (n=3) and 12-hr tumor targeting (n=4) studies. (FIG. 12B) Ex vivo fluorescence (FL) and bright field (BF) imaging of tumors at 12 hr p.i. (FIG. 12C) Haemotoxylin and eosin (H&E)- and silver-stained tumor tissues treated with DOX@AuNPs and NRC-DOX@AuNPs at 12 hr and 3 days p.i., respectively. Scale bar, 100 μm. (FIG. 12D) Analysis of the penetration distance of silver-stained AuNPs from the nearest blood vessels on 3 days p.i. Determination of vessel diameter and vessel-to-vessel distance was in FIGS. 14A-B. Box indicates median and 25-75% interquartile range. ****P<0.0001. (FIGS. 12E-G) Analysis of whole-tumor delivery of the renal clearable AuNPs. H&E- and silver-stained tumor tissues showed the tumor morphology (FIG. 12E) and NP distribution (FIG. 12F). Analysis of the NP accumulation in whole tumor (˜8 mm in diameter) was achieved by collecting the silver-stained counts (per 0.2 mm²) in the inner tumor, peripheral tumor and intermediate areas, where no significant difference was found among three defined regions (FIG. 12G). Scale bar, 500 μm. Box indicates median and 25-75% interquartile range.

FIGS. 13A-B. Vessel diameter and vessel-to vessel distance in MCF tumor. (FIG. 13A) The diameters of tumor vessels were measured based on the length and width of each vessel (yellow lines) and the vessel-to-vessel distance were measured from the nearest distance from one vessel to another (dashed black arrows). Scale bar, 100 μm. (FIG. 13B) Analysis of the vessel diameter and vessel-to-vessel distance. The mean vessel diameter is 32.6 μm and the mean vessel-to-vessel distance is 202.1 μm, which were similar to reported results.^{23,24} Box indicates median and 25-75% interquartile range.

FIG. 14. Tumor accumulation and cancer cell uptake of DOX@AuNPs. H&E- and silver-stained tumor tissues treated with DOX@AuNPs on 3 days p.i. The AuNPs were found to distribute all over the tumor tissue and many AuNPs were taken up by tumor cells (Scale bar, 20 μm for inset), indicating the interaction of DOX@AuNPs with tumor cells and successful delivery. Scale bar, 50 μm.

FIGS. 15A-D. Intratumoral penetration of loaded drug for the renal clearable and non-renal clearable DDSs. (FIG. 15A) Confocal fluorescence microscopy imaging of the tumor microenvironment with 12 hrs post-injection of DOX@AuNPs and NRC-DOX@AuNPs. Full-scale width=320 mm. (FIGS. 15B-D) Quantification of drug diffusion based on the cell count interacting with DOX across different distances from tumor blood vessels. As shown in (FIG. 15B) and (FIG. 15C), starting from the vessel center, the distances were defined as R1 (30), R2 (60), R3 (90) and R4 (120 mm), and different regions of interest (ROIs, A1, A2, A3, A4) were defined for each distance range. The cells with or without DOX interaction were counted based on the nuclei (DAPI) and DOX signals within each ROI for DOX@AuNPs or NRC-DOX@AuNPs, for further plotting in (FIG. 15D) (n=10). Scale bars=50 mm (FIG. 15C). For FIG. 15D, *P<0.05, **P<0.01, ***P<0.005, ****P<0.0001 (One-Way ANOVA within the DOX@AuNPs group or NRC-DOX@AuNPs group).

FIGS. 16A-C. Targeting and penetration of renal clearable DOX@AuNPs in metastatic tumors in brain. Tumor metastasis in brain was developed 2 weeks after the intravenous injection of 4T1 cancer cells into balb/c mice. The 5 nm DOX-loaded AuNPs (DOX@AuNPs) was used for understanding the targeting and permeation of the DDS in brain metastatic tumors, together with the free DOX and the 90 nm Doxil. The metastatic tumors were evident as the clusters of nuclei after fluorescence staining. After 12 h intravenous injection, the free DOX showed negligible accumulation and retention in the metastatic tumors (FIG. 16A); whereas, the significant extravasation and accumulation of DOX in tumor was found for the administrated DOX@AuNPs (FIG. 16B). In contrast, the 90nm Doxil showed high concentration in blood vessel (FIG. 16C), however, no observable drug extravasation and accumulation into tumor sites was found.

FIGS. 17A-G. Therapeutic efficacy of the DOX@AuNPs and NRC-DOX@AuNPs. (FIGS. 17A-D) Normalized tumor growth curves and survival rates of the MCF-7 tumor-bearing nude mice (FIGS. 17A-B) and 4T1 tumor-bearing balb/c mice (FIGS. 17C-D) during successive treatments. The MCF-7 tumor cells were implanted subcutaneously on day 0, the mice were treated with different formulas on day 8, 11, 14, 17, and 20. * indicates P<0.05, <0.005 and <0.0005 for DOX@AuNPs versus NRC-DOX@AuNPs, free DOX and PBS, showing significant differences. ** indicates P<0.005, <0.001, <0.0005 for DOX@AuNPs versus NRC-DOX@AuNPs, free DOX and PBS, respectively (Kaplan-Meier). To be noted, the AuNPs without DOX loading showed no tumor inhibition. (FIGS.17E-G) DOX@AuNPs showed significant inhibition of lung metastasis of breast cancer based on the nodule counts (FIG. 17F) and sizes (FIG. 17G) of the metastatic lung tumors after the successive treatments (day-23). Scale bar, 1 mm.

FIG. 18. Normalized MDA-MB-231 tumor growth curves with successive treatments of different formulas. The treatments were at an equivalent dose of DOX, 5 mg/kg body weight, every 3-day for 5 times. Similar to the other two breast cancer models (MCF-7 and 4T1), DOX@AuNPs showed improved antitumor efficacy in comparison with NRC-DOX@AuNPs and free DOX. To be noted, the MDA-MB-231 tumor xenograft showed a slower growth rate compared to MCF-7 and 4T1 breast cancer models.

FIGS. 19A-B. Efficacious doses of DOX@AuNPs, and free DOX. (FIG. 19A) Mice tumor growth with successive treatment of DOX@AuNPs (0.75 mg/kg), free DOX (5 mg/kg) and PBS (n=5). Low-dose treatment of DOX@AuNPs (0.75 mg/kg) showed comparable tumor inhibition to treatment of DOX (5 mg/kg), indicating the significant enhanced targeting and efficacy. The treatments were conducted on day 8, 11, 14, 17, 20 days post-implantation. (FIG. 19B) Body weight change during the therapeutic study (n=5). The treatment of DOX@AuNPs showed no significant body weight loss.

### DETAILED DESCRIPTION OF EXEMPLARY EMBODIMENTS

To address the fundamental and practical challenges raised above regarding drug delivering nanoparticle functionality, the inventors designed a specific gold nanoparticle (AuNP) based drug delivery system (DDS), which has the following characteristics that conventional DDSs do not possess. The invented AuNP-based DDS can be readily loaded with a large number of chemodrugs through adsorption or chemical conjugation while maintaining its overall hydrodynamic diameters below 10 nm, preventing serum protein binding to the loaded chemodrugs, enhancing chemodrug elimination through the kidneys, decreasing the massive drug accumulation in and acute toxicity to liver as well as shortening the retention of chemodrugs in the normal tissues and vital organs. In this application, a well-known chemodrug, doxorubicin (DOX), will be used as drug model to demonstrate the invention and applicability. DOX has been widely used in the evaluation of different DDSs and free DOX is known to bind serum protein rapidly and also be retain in normal tissues for long time, resulting in not only low efficacy but high toxicity to heart, liver, kidney, and healthy tissues. As control, one-order larger, 30-nm AuNPs with the same surface chemistry, free DOX and DOXIL were also investigated under the same conditions; so that how the in vivo transport (targeting and clearance) and therapeutic efficacy of renal clearable drug-AuNP (e.g., DOX-AuNP) constructs can be quantitatively unravelled.

By precise control of AuNP size and specific design of surface chemistries (incorporating PEG ligands and hydrophobic aromatic ligands such as mercaptobenzoic acid at a ratio of 1:1.5), they designed specific DOX-AuNP constructs loaded with a large number of DOX while the constructs can still have high resistance to serum protein binding and desire physiological stability. In this way, renal clearance of “off-target” DOX was enhanced 3.6 times over that of free DOX (FIG. 1A) and liver accumulation of DOX was reduced nearly one order (FIG. 1B). In addition, the obtained drug-AuNP constructs held minimal interactions with and short retention in normal tissues and organs such as muscle, heart, and lung, etc. (FIG. 1C). Therefore, the maximal tolerated dose (MTD) of DOX was improved with the renal clearable delivery system. While the renal clearable DDS is one-order smaller than non-renal clearable DDS, they can still target tumor tissues and deliver DOX with an efficiency two times greater than the 30-nm AuNPs (FIG. 1D). More importantly, ultrasmall renal clearable DOX-AuNP constructs exhibited much higher tumor penetration than the larger compartments. Because of these strengths, the therapeutic index of drug was improved by one order with the treatment of the as-designed renal clearable drug-AuNP constructs. These new findings indicate that ultrasmall renal clearable drug-AuNP constructs can take different in vivo transport kinetics to respond to normal and cancerous environment. This transport behavior of the renal clearable drug-AuNP constructs opens a new path to targeting tumor tissues rapidly at high concentration and deep penetration while minimizing side effects of anticancer drugs in vivo, which is expected to expedite clinical translation of cancer nanomedicines.

The successive loading and delivery of many other drugs molecules were also investigated and discussed in detail. For the clinically approved anticancer drugs, the anthracycline and platinum-based drugs (such as cisplatin and oxalipatin), as well as the commercially available drugs with the most liver toxicity, can all be loaded onto the renal clearable AuNPs after specific modification on nanoparticle surface. Meanwhile, the clinically approved fluorescent imaging probes as indocyanine green (ICG) and methylene blue (MB) as well as the gadolinium contrast agent for magnetic resonance imaging (MRI) can also be loaded onto the renal clearable DDS. For the molecules with large molecular weight (m.w.), such as small interfering RNA (siRNA, with m.w.˜16000, for example) can be loaded as well, which will be further discussed in the loading studies.

Not limited to the orthotopic breast cancer models (such as MCF-7 and triple negative breast cancer, MDA-MB-231, xenograft), the present renal clearable drug-AuNP constructs can effectively treat other cancer types as well, such as primary and metastatic breast cancer (murine cancer 4T1) and metastatic brain tumors.

These and other aspects of the disclosure are set out in detail below.

### I. NANOPARTICLES

The claimed disclosure provides nanoparticle compositions comprising gold nanoparticles, methods for preparing the nanoparticle compositions and methods of using the nanoparticle compositions. As used herein, the term “nanoparticle” refers to an association of 2-1000 atoms of a metal, in this case gold. Nanoparticles may have diameters in the range of about 0.5 to about 5 nm. As used herein, diameter refers to the core size of the metal core. The metal core may be a gold core (i.e., only gold, predominantly gold, or partially gold). The nanoparticle metal may also comprise, consist of or consist essentially of silver, copper, platinum. In other preferred embodiments, the nanoparticles comprise approximately 2-10000, approximately 2-1000, approximately 2-500, approximately 2-250, approximately 2-100, approximately 2-25 atoms, or approximately 2-10 atoms. The nanoparticles may also be luminescent and emit one, two or more different colored emissions ranging from blue to infrared.

The engineered nanoparticles carry unique surface modifications that permit high drug loading capacity and stability. These surface modifications are designed in the following ways: 1) they need enable nanoparticles to suspend well in physiologic environments and exhibit high physiologic stability; 2) they enable the nanoparticles highly resistant to serum protein adsorption; 3) these unique surface chemistries allow anticancer drugs to be loaded through non-covalent interactions such as pi-pi stacking (also called π-π stacking) or electrostatic interactions; 4) These unique surface chemistries need to enable drugs to be loaded onto the particles more than 5 drug molecules per particle. Surface ligands include but are not limited to benzyl mercaptan (benzomercaptan), mercaptobenzoic acid, aminothiophenol, methylbenzenethiol, thiolated pyrene, thiolated nucleobases; 5) unique surface chemistries enable the nanoparticles to be cleared out of the body through the urinary system. In some embodiments of the present disclosure, greater than 30%, 40%, 50%, 60%, 70%, 80% or 90% of the nanoparticles may be eliminated from the body through the urinary system.

The nanoparticles will further comprise a therapeutic agent. Significantly, the drug will be associated with the surface of the nanoparticle via non-covalent attachment, such as by pi-pi stacking of the drug and a surface component, such as benzyl mercaptan (benzomercaptan), mercaptobenzoic acid, aminothiophenol, methylbenzenethiol, thiolated pyrene, etc. Pi-pi stacking refers to attractive, noncovalent interactions between aromatic rings, since they contain pi bonds. These interactions may be important in nucleobase stacking within DNA and RNA molecules, protein folding, template-directed synthesis, materials science, and molecular recognition. Despite intense experimental and theoretical interest, there is no unified description of the factors that contribute to pi-pi stacking interactions.

Finally, they exhibit enhanced tumor targeting efficiency, and reduced the non-specific accumulation in normal tissues and organs due to rapid elimination through the urinary system.

Benzyl mercaptan is an organosulfur compound with the formula C₆H₅CH₂SH. It is a common laboratory alkylthiol that occurs in trace amounts naturally. It is a colorless, malodorous liquid. It can be prepared by the reaction of benzyl chloride and thiourea. The compound has been used as a source of the thiol functional group in organic synthesis.

In certain embodiments, the surface of the nanoparticle may also be coated with other materials. In certain embodiments of the disclosure, the material is an anti-fouling ligand, which is a zwitterionic material such as sulfobetaine methacrylate (SBMA), carboxybetaine methacrylate (CBMA), poly(carboxybetaine acrylamide) (polyCBAA) or a mixed charge material. In certain embodiments of the disclosure the material is glutathione, cysteine, cysteine-glycine, cysteine-glutamate, and other thiolated surface ligands which can be protonated at different pHs.

In certain embodiments, the ligand (or a further ligand) is capable of binding to at least one cellular component. The cellular component may be associated with specific cell types or having elevated levels in specific cell types, such as cancer cells or cells specific to particular tissues and organs. Accordingly, the nanoparticle can target a specific cell type, and/or provides a targeted delivery for the treatment and diagnosis of a disease. As used herein, the term “ligand” refers to a molecule or entity that can be used to identify, detect, target, monitor, or modify a physical state or condition, such as a disease state or condition. For example, a ligand may be used to detect the presence or absence of a particular receptor, expression level of a particular receptor, or metabolic levels of a particular receptor. The ligand can be, for example, a peptide, a protein, a protein fragment, a peptide hormone, a sugar (i.e., lectins), a biopolymer, a synthetic polymer, an antigen, an antibody, an antibody fragment (e.g., Fab, nanobodies), an aptamer, a virus or viral component, a receptor, a hapten, an enzyme, a hormone, a chemical compound, a pathogen, an aromatic compound, a microorganism or a component thereof, a toxin, a surface modifier, such as a surfactant to alter the surface properties or histocompatability of the nanoparticle or of an analyte when a nanoparticle associates therewith, and combinations thereof.

Other metals such as silver, copper, and platinum and even carbon, etc., may be used to generate nanoparticles for use in accordance with the present disclosure.

Drugs for attachment to nanoparticles in accordance with the present disclosure include antibiotics, antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, extracellular matrix components, vasodialators, thrombolytics, antimetabolites, growth factor agonists, antimitotics, statin, steroids, steroidal and nonsteroidal anti-inflammatory agents, angiotensin converting enzyme (ACE) inhibitors, free radical scavengers, PPAR-y agonists, small interfering RNA (siRNA), microRNA, anti-cancer chemotherapeutic agents, photothermal agents, photodynamic agents, photoacoustic agents and immunotherapeutic agents.

A “chemotherapeutic agent” or “chemodrug” is defined as a compound or composition that is administered in the treatment of cancer. These agents or drugs are categorized by their mode of activity within a cell, for example, whether and at what stage they affect the cell cycle.

Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin yi and calicheamicin on; dynemicin, including dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone;

etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine;

novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

### II. PREPARING NANOPARTICLES

The nanoparticles need to be designed in a specific way to meet the requirements of enhancing tumor targeting while accelerating the clearance of drugs from normal tissue and normal organs through the kidneys. Such approaches may be used to synthesize ultrasmall gold nanoparticles with thiolated ligands such as amphiphilic ligands (poly(ethylene glycol)) and zwitterionic ligands, etc. Additionally, the ultrasmall nanoparticles may be synthesized by reduction of chloroauric acid in aqueous solution with (or without) the presence of reducing agent as sodium borohydride (NaBH₄), sodium cyanoborohydride, formaldehyde, tetra-n-butylammonium bromide (TBAB), Tris-2-carboxyethylphosphine hydrochloride (TCEP), ascorbic acid, formic acid, oxalic acid, reducing sugars, or compounds with similar reducing abilities, etc.

Additionally, the specifically designed AuNPs may be prepared using the following protocol: the HAuCl₄ and thiol ligands (for instance, poly(ethylene glycol) methyl ether thiol) in solution (typically, in the range of 0.01 to 10 mM) with the gold-thiol ratio from 1:3 to 3:1 (this ratio varies with different reducing agents) are mixed and reduced. In addition, the thiolated

AuNPs will also be synthesized in solution via the heating process with an oil bath at elevated temperature (the temperature varies from 45 to 95° C. for 1 to 24 h). The resulting solution will be cooled to room temperature and centrifuged to remove larger nanoparticles. The resulting supernatant will be dried and collected as powder. Since PEG-AuNPs cannot stably load enough drugs, additional surface modification is also crucial for the preparation of drug-AuNP constructs. To do that, PEG-AuNPs and second thiolated conjugation ligands will be incubated under vortex at room temperature and the dual-ligand AuNPs are purified with centrifugal filter for removing free ligands. In some embodiments, the ratio of the noble metal nanoparticle to the ligand is about 1:100 to about 1:10,000. The ratio of second ligand to PEG on the particle is about 1:0.25 to 1:4. After incubating these components together for a period of time from about 1 to 72 hrs, the modified nanoparticles will be then purified by centrifugal filter. The adsorption of second ligand is confirmed using nuclear magnetic resonance, elemental analysis or other appropriate spectroscopic techniques.

To prepare drug-AuNP constructs, the as-synthesized dual-ligand AuNPs and therapeutic molecules will be mixed in aqueous solution and incubated under vortex, and then purified by size exclusion column. In some embodiments, the ratio of the modified nanoparticle to the therapeutic drug is about 1:1 to about 1:10000 for drug loading.

### III. PHARMACEUTICAL FORMULATIONS

Where clinical applications are contemplated, it will be necessary to prepare pharmaceutical compositions in a form appropriate for the intended application. In some embodiments, such formulation with the nanoparticles of the present disclosure is contemplated. Generally, this will entail preparing compositions that are essentially free of pyrogens, as well as other impurities that could be harmful to humans or animals.

One will generally desire to employ appropriate salts and buffers to render the nanoparticles stable and allow for uptake by target cells. Buffers also will be employed when recombinant cells are introduced into a patient. Aqueous compositions of the present disclosure comprise an effective amount of the nanoparticles to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The nanoparticles may also be administered parenterally or intraperitoneally. Solutions of the nanoparticles as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active nanoparticles in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the nanoparticles described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 mL of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the appropriate regulatory agencies for the safety of pharmaceutical agents.

### IV. METHODS OF THERAPY

In an embodiment of the disclosure, the therapeutic agent attached to the nanoparticle permits use of the nanoparticle in a therapy, such as for cancer. As such, an embodiment of the disclosure is directed to a method of targeting/treating a disease site, such as a tumor, comprising administering an effective amount of a gold nanoparticle composition as described above and comprising at least one therapeutic agent.

In particular, compositions that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces death of cancerous cells (e.g., induces apoptosis of a cancer cell), as assayed by identifying a reduction in hematological parameters (complete blood count—CBC), or cancer cell growth or proliferation. In some embodiments, amounts of the compound used to induce apoptosis of the cancer cells is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Addtionally, the compound may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

A. Cancers and Hyperproliferative Disease

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the nanoparticles described herein may be used to lead to decreased cell counts and as such can potentially be used to treat a variety of types of cancer lines. In some aspects, it is anticipated that the nanoparticles may be used to treat virtually any malignancy.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous hi stiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

B. Combination Therapies

It is envisioned that the compound described herein may be used in combination therapies with one or more cancer therapies or a compound which mitigates one or more of the side effects experienced by the patient. It is common in the field of cancer therapy to combine therapeutic modalities. The following is a general discussion of therapies that may be used in conjunction with the therapies of the present disclosure.

To treat cancers using the methods and compositions of the present disclosure, one would generally contact a tumor cell or subject with the compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the compound described herein may precede or follow the other treatment by intervals ranging from minutes to weeks. One would generally ensure that a significant period of time did not expire between the times of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where the compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. The following is a general discussion of cancer therapies that may be used combination with the compounds of the present disclosure.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents that can be used in combination according to the present disclosure include the use of two nanoparticle preparations having different chemotherapeutic agents, or a single nanoparticle preparation combined with a standard chemotherapy regimen. The list of chemotherapeutic agents set out above is included here by reference.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given.

This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p9′7), gp68, TAG-72, HMFG, Sialyl Lewis Antigen, MucA,

MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF and TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believed to be particularly efficacious in reducing the recurrence of the tumor. Additionally, the compounds of the present disclosure can also be used in a neoadjuvant setting.

5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1 (3, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents may be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 41.1° C.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the appropriate pharmaceutical agent regulatory agencies.

It also should be pointed out that any of the foregoing therapies may prove useful by themselves in treating cancer.

### V. EXAMPLES

The following examples are included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Preparation and characterization of DOX@AuNPs and NRC-DOX@AuNPs. Renal clearable gold nanoparticle (AuNP)-based drug delivery system was designed in a following way. AuNPs with core size below 6 nm were synthesized using thermal or chemical reduction method. The AuNPs are coated with two types of surface ligands: One ligand should be antifouling ligands such as zwitterionic ligands such as glutathione, or PEG ligands that can prevent serum protein adsorption while maintaining the overall hydrodynamic diameter below 10 nm; the secondary ligand should be hydrophobic aromatic and charged ligands that allow drug molecules adsorbed through pi-pi or electrostatics interactions. Following is an example. The renal clearable PEG-AuNPs were synthesized and further modified with 4-Mercaptobenzoic acid (MBA) in ethanol at room temperature and the dual-ligand AuNPs were purified with centrifugal filter for removing free ligands. To prepare the DOX-AuNP constructs, DOX@AuNPs, the as-synthesized dual-ligand AuNPs and DOX were mixed at a mass ratio of 5-to-2 in ultrapure water and incubated overnight under vortex, and then purified by size exclusion column (Sephadex LH-20). The obtained DOX@AuNPs solution was further concentrated with centrifugal filter (MWCO, 30 kDa). The non-renal clearable PEG-AuNPs (NRC-PEG-AuNPs) were synthesized via solid-state thermal reduction of gold salt with glycine as reported previously (Peng et al., 2017), followed by the incubation of AuNPs with 10 mM PEG-SH for surface coating of PEG ligands. For surface modification, NRC-PEG-AuNPs (2 mg/mL) and MBA (0.25 mg/mL) were incubated in 0.01 M sodium tetraborate for 30 min under vortex. The resulting NPs were purified and washed with centrifugal filter before drug loading. To prepare NRC-DOX@AuNPs, the as-modified NRC-AuNPs and DOX (1:1, w/w) were incubated in water over night and purified using centrifugal filter (MWCO, 100 kDa). The absorbance spectra were collected using a Varian 50 Bio UV-Vis spectrophotometer. Quantification of DOX loading was achieved by the UV-Vis spectroscopy of DOX@AuNPs and free DOX (characteristic absorbance at 350 and 480 nm), as well as ICP-MS measurement. Transmission electron microscopy (TEM) images were taken by a 200 kV JEOL 2100 transmission electron microscope. HDs were analysed using a Brookhaven 90 Plus dynamic light scattering particle size analyser. Nuclear magnetic resonance (NMR) spectra were acquired with Bruker 600 MHz NMR spectrometer.

Animal and tumor xenograft model. The animal studies were conducted by following the guidelines of the University of Texas System Institutional Animal Care and Use Committee. The CD-1 and nude mice were housed in ventilated cages under standard environmental conditions (23±1° C., 50±5% humidity and a 12/12 hr light/dark cycle) with water and standard laboratory food. The MCF-7 cancer cells were cultured in Dulbecco's modified eagle medium (DMEM) with supplements (10% fetal bovine serum and 1% penicillin-streptomycin) at 37° C. in humidified atmosphere containing 5% CO₂. For tumor xenograft model, suspension of MCF-7 cells (DMEM: Matrigel, 2:1 (v/v), containing ˜1×10⁶ per 100 μL) were injected subcutaneously near the mammary fat pat (MFP) area. The tumors were allowed to develop for 1 w before imaging, biodistribution or therapeutic studies.

Clearance, physiological stability, and elimination. To quantify in vitro stability of DOX@AuNPs, the AuNPs (5 μM) and human serum albumin (HSA, 5 μM) in PBS (pH 7.4) were incubated at 37° C. for 3 hr before HD measurement. In order to quantify the renal clearance of DOX@AuNPs, female CD-1 mice (6-8 week old) were injected intravenously with DOX@AuNPs, NRC-DOX@AuNPs and free DOX, respectively, and the urine was collected and weighed at different time intervals within 24 hr p.i., respectively. To quantify DOX in the urine, sodium acetate (0.05 M, pH 4) was added into the urines for dilution followed by the incubation for 30 min. The suspension was then centrifuged at 21,000 g for 10 min and supernatant was collected and analysed using fluorescence HPLC with a Cis column (Kinetex 5 μm, 150×4.6 mm, Phenomenex) as the stationary phase and sodium acetate buffer/acetonitrile (70: 30, v/v) as mobile phase (Tran et al., 2014; Alhareth et al., 2012). Since DOX is highly fluorescent, the fluorescence signals were collected at 610 nm with excitation at 470 nm. To quantify AuNP in the urine, the urine was dissolved in aqua regia and diluted properly for ICP-MS measurement. To quantify DOX biodistribution at 1 and 24 hr p.i., the organs and tissues were weighed and homogenized with lysis buffer, and homogenated samples were added with 10% Triton X-100, deionized water and acidified isopropanol (0.75 N HCl) (1:2:7, v/v) and mixed well via vortex (Gabizon, 1992; Laginha et al., 2005). Extraction was performed at −20° C. for overnight and supernatant was obtained after centrifugation at 15,000 g for 10 min. The DOX concentrations were quantified based on fluorescence analysis. Standard addition of free DOX was performed before homogenization for mice tissues with PBS treatment. For AuNP biodistribution, the mice were sacrificed at selected time-point, and main organs were collected, weighed and dissolved in aqua regia before ICP-MS measurements. For pharmacokinetic study, the DOX concentrations in blood were analysed using HPLC with the same approach described above.

Toxicity studies. To quantify the acute toxicity, the inventors intravenously injected DOX@AuNPs, NRC-DOX@AuNPs, equivalent DOX (5 mg/kg, ×5) and PBS in 15 days into female CD-1 mice (6-8 week old), of which body weight and physical abnormalities were recorded every 2 days. For blood chemistry study, the blood was collected through cardiac puncture 15 days p.i. and kept on ice for 30 min for coagulation. Then, the serum was acquired and analysed after centrifugation at 12,000 g for 15 min with removal of coagulated blood cells. The organs and tissues were also collected, fixed in formalin (10%) and embedded in paraffin before H&E staining and pathological evaluation. In order to determine the MTD of DOX@AuNPs, NRC-DOX@AuNPs and free DOX, the female CD-1 mice were injected intravenously with single dose of DOX@AuNPs, DOX at different concentrations, and then the body weight was monitored within 10 days. The similar studies were also conducted using PBS as a control. The mice were sacrificed on 10 days p.i. and the blood were extracted via cardiac puncture. The serum samples were acquired after centrifugation of whole blood at 10,000 g for 15 min for further blood analysis.

Tumor targeting, penetration and antitumor efficacy studies. In order to quantify the targeting efficiency of DOX before and after being loaded onto renal clearable-and non-renal clearable AuNPs, MCF-7 tumor-bearing nude mice were injected intravenously with DOX@AuNPs, NRC-DOX@AuNPs, equivalent DOX (5 mg/kg) and PBS, respectively, and sacrificed at 12 hr p.i. The tumors were collected and homogenized and DOX concentrations were measured using the same approaches described in the biodistribution studies. The ex vivo tumor fluorescence imaging was acquired using a Carestream Molecular imaging system In-Vivo FX PRO with an excitation filter of 470±10 nm and an emission filter of 600±20 nm. To quantify the NP tumor penetration, the inventors intravenously injected MCF-7 tumor-bearing nude mice with DOX@AuNPs and NRC-DOX@AuNPs, respectively. The mice were sacrificed 12 hr and 3 days p.i. respectively. Tumor tissues were fixed, embedded in paraffin and sliced before haematoxylin and eosin (H&E), and silver staining. To enable visualization of AuNPs in the tumor issues, dewaxed tumor slides were incubated with silver nitrate (0.1 M) and hydroquinone (2 mM) for 15 min at room temperature, followed by washing with pure water and haemotoxylin and eosin staining (Du et al., 2017). The colour images of the stained slices were taken using Nikon SMZ-18 stereomicroscope (13.5×). In order to evaluate therapeutic efficacy, the tumor-bearing nude mice were injected successively with DOX@AuNPs, NRC-DOX@AuNPs, equivalent DOX (5 mg/kg), equivalent AuNPs, and PBS as a control on day 8, 11, 14, 17 and 20 post-implantation. The length (L) and width (W) of the tumors were measured with a digital calliper and the volume (V) was calculated by the equation V=½ LW². The body weights were also monitored during the treatment. The mice were euthanized and sacrificed at the end of the study (day 23) and tumor tissues were collected. For survival study, the tumor bearing mice were treated same with therapeutic study, and tumor sizes were monitored. The mice with tumor sizes over 1,000 mm³ were euthanized (MacKay et al., 2009). The silver-stained tumor samples were imaged to visualize the NPs in tumor microenvironment. To establish the metastatic lung cancer model, female Balb/c mice were implanted with 4T1 mammary cancer cells subcutaneously and the cancer cells would be readily developed as lung metastatic nodules after a week. The tumor-bearing mice were treated with DOX@AuNPs, NRC-DOX@AuNPs, DOX, Doxil as well as PBS (5 mg/kg DOX equivalent, on day 8, 11, 14, 17, 20) and the mice were sacrificed on day 23 and the metastatic tumor nodules in the lungs were stained with DAPI for nodule count and size measurement.

### Example 2

### Results

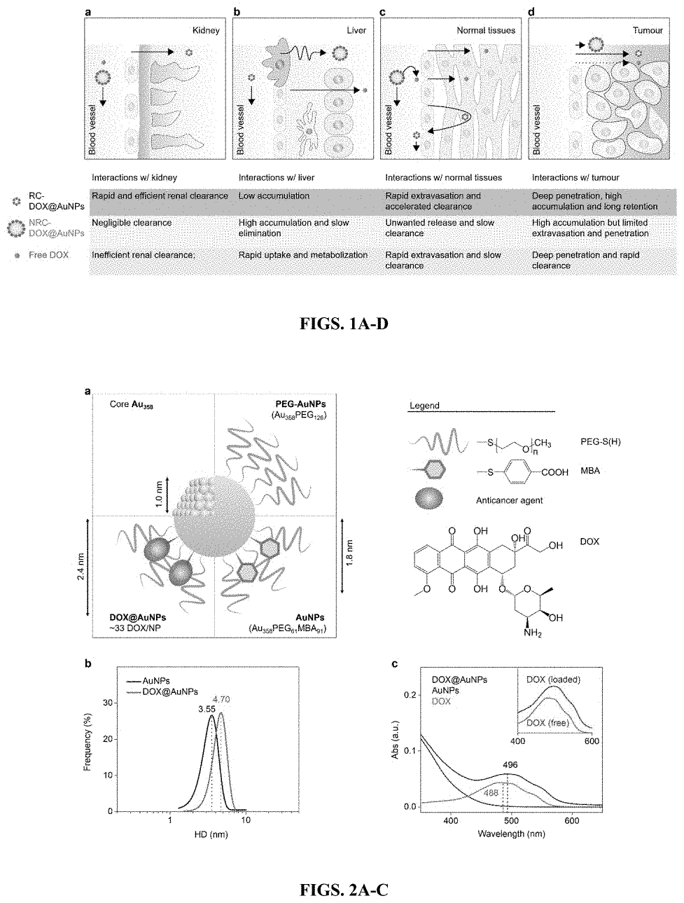

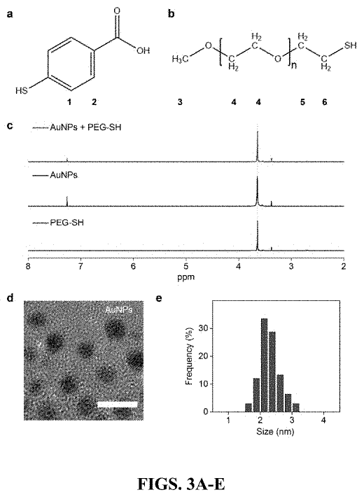

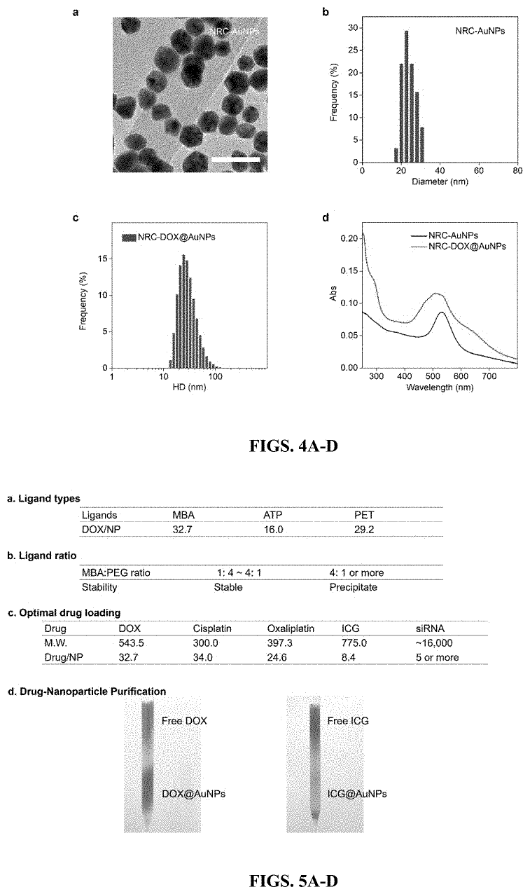

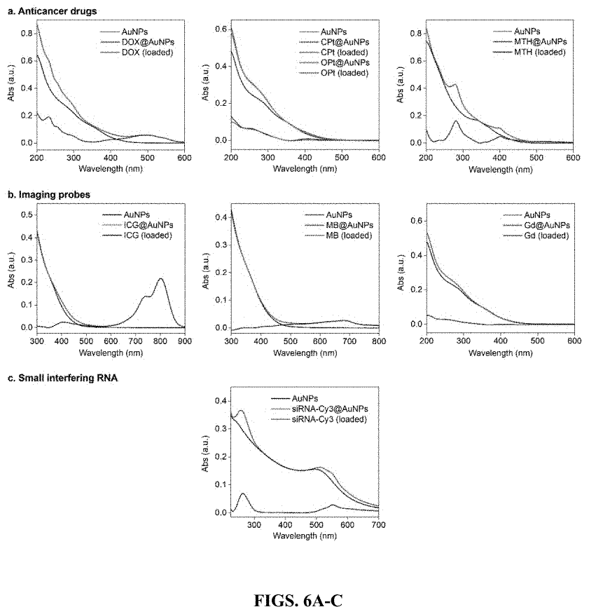

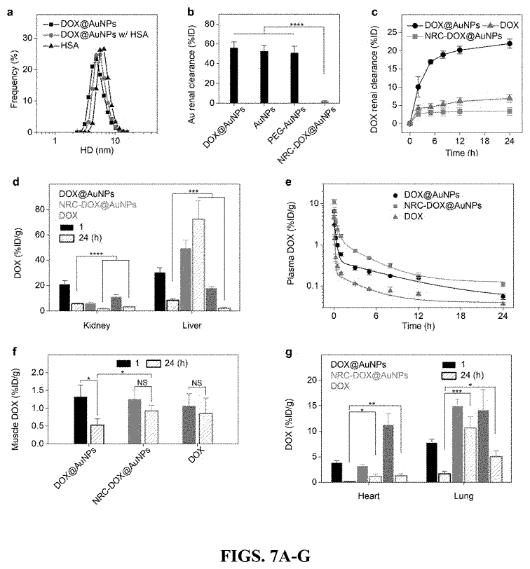

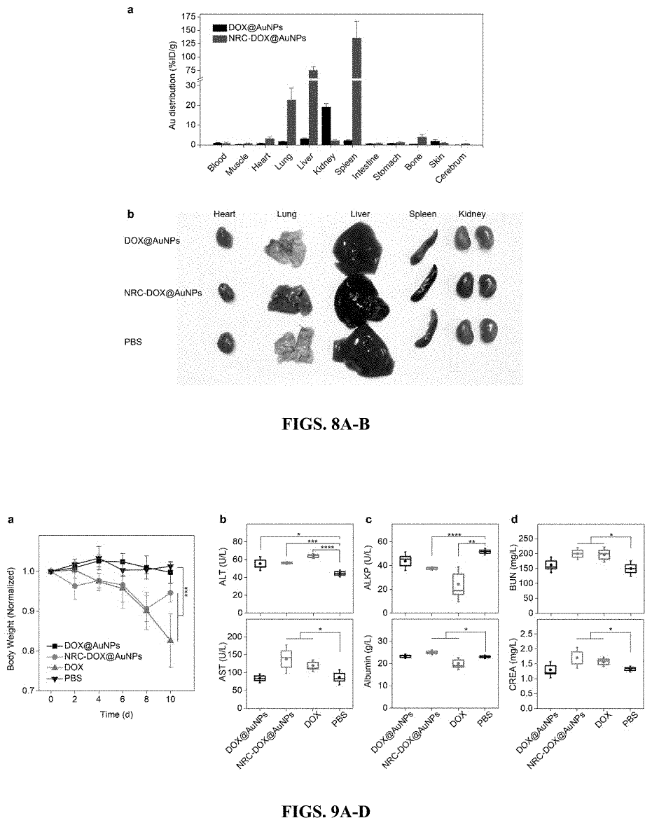

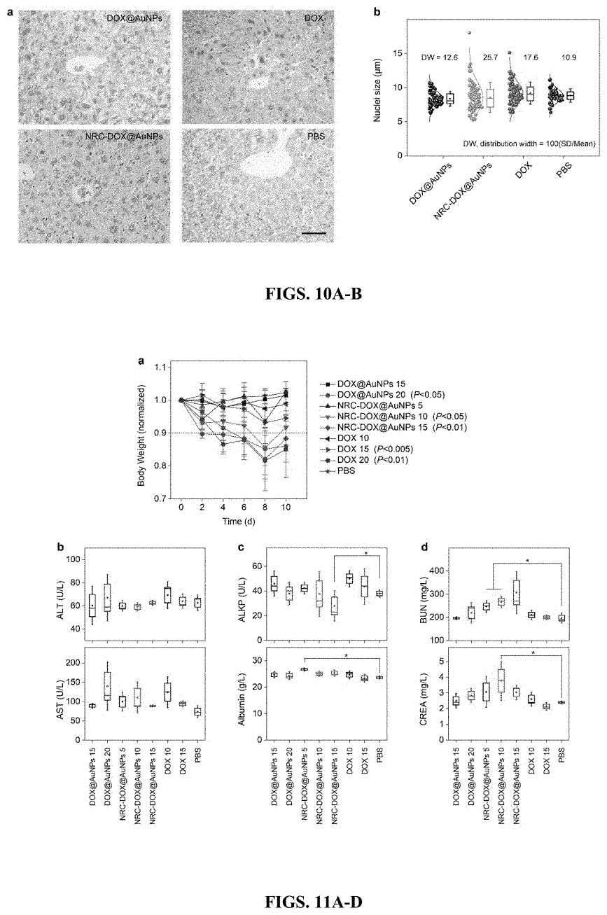

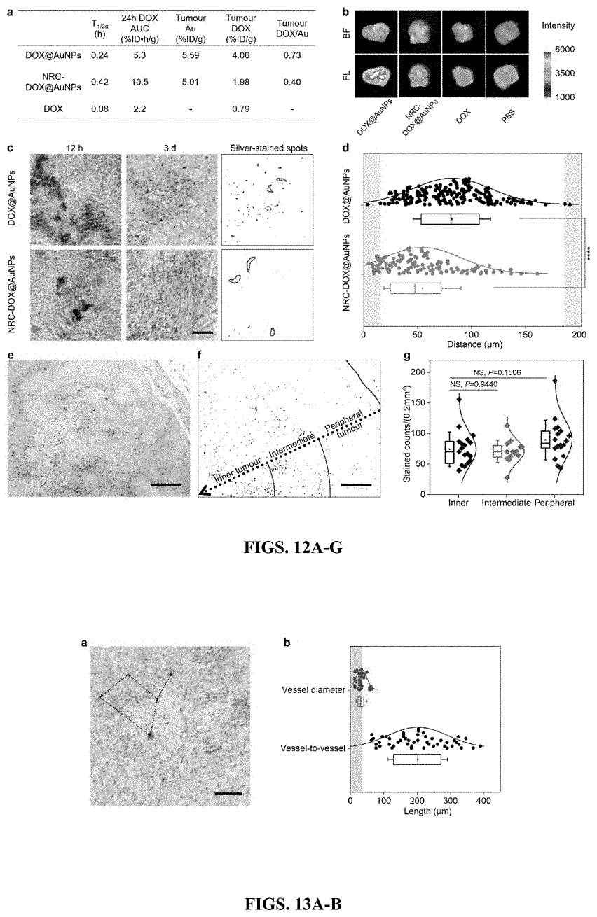

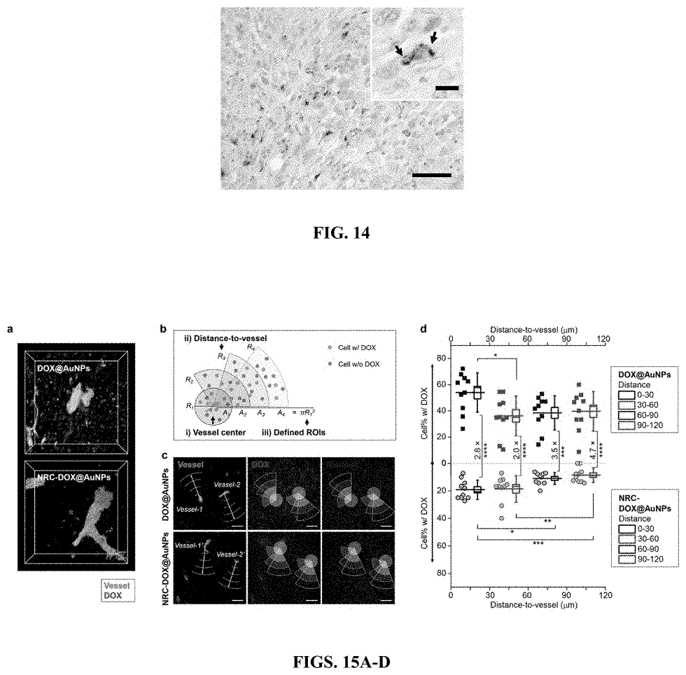

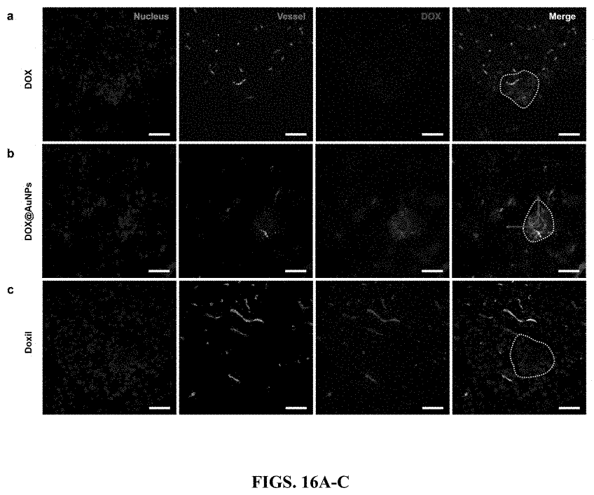

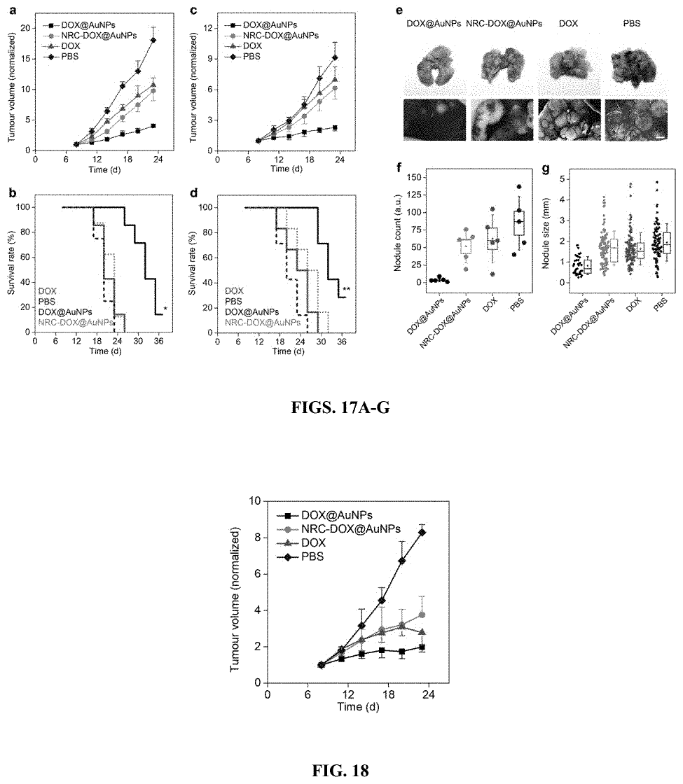

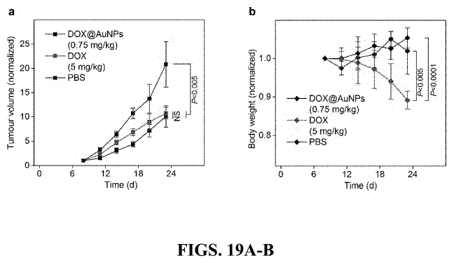

Loading DOX onto renal clearable PEGylated AuNPs (DOX@AuNPs). To resolve the problem of inefficient loading efficiency for ultrasmall DDSs, the inventors incorporated a secondary ligand, 4-mercaptobenzoic acid (MBA), onto the 5-nm PEG-AuNPs through ligand exchange reaction (FIG. 2A); so that more DOX could be loaded on the particle through π-π interactions between DOX and the aromatic ligands (MBA) (Shi et al., 2015). The obtained dual-ligand AuNPs (Au358PEG61MBA91, FIGS. 3A-E) indicated not only PEG ligands were partially replaced by MBA but also more MBA molecules were adsorbed onto the AuNPs. As a result, the loading capacity of the as-modified AuNPs was increased nearly five times to 32.7±6.8 DOX/NP, much higher than those of other known ultrasmall NPs with comparable sizes (typically 5-25 DOX/NP) (Liao et al., 2014; Lee et al., 2006; Zhang et al., 2010). The DOX-AuNP constructs (DOX-loaded PEG/MBA-AuNPs, DOX@AuNPs) showed a 1.2-nm increase in HD compared to free AuNPs (4.70±0.89 versus 3.55±0.77 nm, respectively, FIG. 2B). In addition, the strong characteristic absorption of DOX at 500 nm was observed, superimposing on the absorption of AuNPs (FIG. 2C). Moreover, an 8-nm redshift in the absorption of the loaded DOX suggested strong dipole-dipole coupling of multiple DOX molecules on the particle surface (Sun et al., 2016). Further increasing drug loading resulted in the instability of DOX@AuNPs; thus, in this study, the inventors mainly focused on the AuNPs loaded with 33 DOX molecules. As a control, they prepared non-renal clearable PEG/MBA-AuNPs with HD of ˜30 nm, which could also be loaded with DOX via similar procedures (FIGS. 4A-D). The average drug loading of the obtained non-renal clearable DOX-loaded AuNPs (NRC-DOX@AuNPs) was 57,900±20,700 DOX/NP.

With further loading study, we found that, all the aromatic ligands, like 4-mercaptobenzoic acid (MBA), 4-aminothiophenol (ATP), 2-phenylethanethiol (PET), could load certain amount of DOX molecules whereas the mercaptobenzoic acid showed the highest capacity (FIG. 5A). In addition, the selected MBA-to-PEG ratios enabled the DOX loading with high efficiency and stability (FIG. 5B). In addition, the loaded drugs vary from clinically used chemodrugs as doxorubicin, platinum-based drugs (cisplatin and oxaliplatin, based on electrostatic interaction), and imaging probes as indocyanine green (ICG) to small-interfering RNA (based on covalent gold-sulfur bonding), indicating the versatile functionality of the drug loading systems (FIG. 5C). The drug-loaded AuNPs could be effectively purified via size exclusion column (Sephadex) for free drug removal (FIG. 5D). The absorbance spectra of drug-loaded AuNPs as well as loaded drugs were listed (FIGS. 6A-C), where the loaded drug types vary from anticancer drugs, to imaging probes and siRNAs.

PEGylated AuNPs loaded with DOX have Reduced DOX-protein interaction and enhanced DOX body clearance. While more than 30 DOX molecules loaded on the PEGylated AuNPs, the DOX-AuNP constructs remain smaller than 6nm and did not induce size increase after the incubation with human serum albumin (HSA, 6.0 nm) in the physiological environment (FIG. 7A), indicating that the renal clearable AuNPs indeed have large capacity of accommodating the small molecules with strong interaction to serum protein while retaining their resistance to serum protein adsorption. After the intravenous administration of DOX@AuNPs in female CD-1 mice, the AuNPs after being loaded with drugs remained renally clearable with an efficiency of 56.0 ±5.9% ID in 24 hr p.i. (post-injection) from ICP-MS analysis, comparable to free dual-ligand AuNPs and PEG-AuNPs (52.7±6.0 and 50.9±6.9%, respectively, FIG. 7B), indicating that drug loading did not alter renal clearance of the AuNPs. The overall renally cleared DOX in 24 hr was enhanced to 22.0±1.3% ID by renal clearable AuNPs (FIG. 7C), which was 3.6- and 6.5-fold higher than those of free DOX (6.1±1.1% ID) and 30 nm NRC-DOX@AuNPs (3.4±1.0% ID), respectively.

The significant enhancement in renal clearance of DOX for DOX-AuNP constructs fundamentally originated from dramatic changes in in vivo transport kinetics of DOX in the kidneys. Unlike conventional non-renal clearable AuNPs that reduced DOX accumulation in the kidneys from 11.4% ID/g to 6.4% ID/g at 1 hr p.i., renal clearable DOX-AuNP constructs increased DOX distribution in the kidneys to 21.4% ID/g (FIG. 7D), indicating that more DOX was transported to the kidneys by the renal clearable AuNPs, consistent with the observed enhancement of DOX renal clearance. On the other hand, due to the strong interactions between 30-nm AuNPs and RES, more than 70% ID/g of DOX accumulated in the liver at 24 h, while only 8.4% ID/g of DOX was found in the liver for the administrated DOX-AuNP constructs. It should be noted that free DOX had even lower accumulation in the liver, which was mainly due to the rapid DOX uptake by hepatocytes and hepatobiliary elimination, which, however, was known to induce liver impairment during the metabolism and excretion. These results were consistent with the pharmacokinetics studies of DOX in the different formulas. The free DOX in the CD-1 mice exhibited rapid plasma elimination (distribution half-life t_{1/2a}=0.08 h, FIG. 7E), consistent with reported results. Meanwhile, both DOX@AuNPs and NRC-DOX@AuNPs showed blood half-lives of 0.24 and 0.42 h, respectively, indicating their renal and hepatobiliary clearance. Overall, 24-h area under curve (AUC) values of DOX@AuNPs (5.3% ID·h/g) and NRC-DOX@AuNPs (10.5% ID·h/g) were 2.4- and 4.8-fold of that of free DOX (2.2% ID·h/g), respectively. These results indicated that using the specific nano-bio interactions of renal clearable AuNPs, the renal elimination of anticancer drugs could be significantly improved.

Renal clearable DOX-AuNP constructs have rapid elimination from normal tissues and vital organs. While the injected DOX@AuNPs were smaller than interendothelial junctions and remained highly permeable into the skeletal muscle (gastrocnemius), the accumulation of DOX in the muscle decreased significantly from 1.32±0.33 to 0.53±0.18% ID/g after 24 hr (60.2% decrease, FIG. 7F). However, for the injected free DOX as well as NRC-DOX@AuNPs, their elimination from muscle was quite slow and not significant (only 19.7% and 26.1% decreases from 1 to 24 h, respectively). Not limited to muscle tissues, renal clearable DOX-AuNP constructs also accelerated the DOX elimination from heart and lung and reduced its accumulation. For the injected DOX@AuNPs, the DOX distributions in heart and lung were only 7.3 and 33.0% of the accumulation of free DOX, and 8.1 and 15.6% of those of NRC-DOX@AuNPs at 24 hr p.i., respectively (FIG. 5G). This significant enhancement in the drug clearance from normal tissues and organs mainly resulted from the minimized interactions of renal clearable AuNPs with the background tissues and organs (FIGS. 8A-B).

Minimizing toxicity and improving tolerance of DOX through enhancing clearance of “off-target” DOX. Renal clearable DOX-AuNP constructs have minimized interactions with plasma, background tissues and organs, resulting in the reduction in the systematic toxicity and improvement in the MTD value of DOX. Acute toxicity study was conducted by successively treating female CD-1 mice with DOX@AuNPs, NRC-DOX@AuNPs, free DOX (equivalent DOX, 5 mg/kg body weight, ×5) and PBS control within 10 d, respectively. Significant weight loss (˜10%) and mortality (25%) of mice were evident from NRC-DOX@AuNPs and free DOX treatment, while the mice treated with DOX@AuNPs retained normal weight during the study (FIG. 9A).

The renal clearable DOX-AuNP constructs also have minimized liver and kidney impairment significantly. The blood chemistry tests showed that free DOX increased alanine aminotransferase (ALT) and aspartate aminotransferase (AST) levels and decreased alkaline phosphatase (ALKP) and albumin levels in serum, which, however, were minimized significantly from the renal clearable DOX-AuNP constructs (FIGS. 9B-C). The NRC-DOX@AuNPs still showed damages to liver function (including elevated serum ALT and AST levels and decreased ALKP level), indicating the toxicity of the NRC-DOX@AuNPs to hepatocytes during the elimination process. In addition, the increase in hepatocyte anisocaryosis and anisocytosis (representing the over-exposure to genotoxic agents) was observed from both NRC-DOX@AuNPs and free DOX treatments from histopathological evaluation (FIGS. 10A-B), which was absent from DOX@AuNPs treatment (Gillet et al., 2000). Moreover, while both free DOX and NRC-DOX@AuNPs were mainly eliminated through the hepatic pathway, they both increased blood urea nitrogen (BUN) and creatinine (CREA) levels in serum (FIG. 9D), indicating that the non-renal clearable AuNPs did not reduce DOX induced nephrotoxicity. However, DOX@AuNPs showed no observable toxicity to kidney even though 3.6-fold more DOX was eliminated through the kidneys. These changes in the levels of the serum biomarkers are clear indicatives of the liver and kidney impairment induced by free DOX as well as NRC-DOX@AuNPs, consistent with reports and clinical observations. In contrast, the absence of these sides effects from DOX@AuNPs treatment clearly suggested that renal clearable DOX-AuNP constructs successively minimized the toxicity of DOX because of accelerated elimination of “off-target” drug molecules out of normal tissues and organs. Further studies showed that the MTD of DOX@AuNPs was increased 50% over free DOX and NRC-DOX@AuNPs (FIGS. 11A-D).

Enhancing tumor targeting of DOX while retaining deep tumor penetration. Renal clearable DOX-AuNP constructs could greatly improve the tumor targeting and penetration compared to free DOX and NRC-DOX@AuNPs. Using nude mice bearing MCF-7 human breast cancer xenograft as model, the inventors investigated the effect of NP-tumor interaction on the delivery of DOX. The tumor targeting of DOX was increased to 4.06±0.38% I D/g by renal clearable DOX-AuNP constructs, 5-time higher than that of free DOX (0.79±0.22% ID/g) at 12 hr p.i. (FIG. 7A). In the meantime, tumor accumulation of the renal clearable AuNPs (in terms of gold) was 5.59±0.43% ID/g from ICP-MS measurement and the ID% ratio of DOX-to-Au was nearly 0.73±0.07, confirming that more than 70% of the AuNPs entered tumors with DOX. On the other hand, 30-nm AuNPs only delivered 1.98±0.42% ID/g of DOX into the tumors even though similar amount of 30-nm AuNPs (5.01±1.19% ID/g) entered the tumors, implying that undesired DOX release of NRC-DOX@AuNPs. While the DOX carried by renal clearable AuNPs was only the half of the NRC-DOX@AuNPs in the plasma (24-hr AUC), the renal clearable AuNPs could still deliver the therapeutic agents to tumor twice as much as the non-renal clearable counterpart, further indicating that renal clearable AuNPs can deliver DOX into the tumors more efficiently than their larger counterparts.

While the limited tumor penetration (via diffusion) has been one major issue for many cancer nanomedicines (Jain & Stylianopoulos, 2010), ultrasmall renal clearable DOX-AuNP constructs have the rapid extravasation and deep tumor penetration. The ex vivo fluorescence imaging studies showed that the whole-tumor DOX intensity for DOX@AuNPs was much higher than those of free DOX and NRC-DOX@AuNPs at 12 hr p.i. (FIG. 12B). The haematoxylin and eosin (H&E)- and silver-stained tumor tissues show that both renal clearable and non-renal clearable AuNPs were distributed close to the tumor vessels 12 hr p.i. (FIG. 12C). However, the renal clearable DOX@AuNPs showed much more significant tumor extravasation and deeper tumor penetration than NRC-DOX@AuNPs on 3 days p.i. By analysing the distance of the stained NPs from the nearest tumor vessels, the inventors found that renal clearable DOX@AuNPs travelled much further from vessels (with median distance of 80.8 μm) while many NRC-DOX@AuNPs still distributed near the tumor vessels (with median distance of 47.5 FIG. 12D). These results were consistent with previous observations that the smaller nanocarriers exhibited much deeper tumor penetration than the larger ones (Perrault et al., 2009; Cabral et al., 2011). Further analysis of the renal clearable DOX@AuNPs (silver-stained spots) in whole tumor showed the NPs exhibited nearly homogeneous distribution from peripheral tumor, through intermediate area, to inner tumor core (FIGS. 12E-G), consistent with the fluorescence tumor images (FIG. 7B). Moreover, the H&E- and silver-stained tumor images showed that many AuNPs were found inside the cancer cells (FIG. 14), further confirming the renal clearable DOX-AuNP constructs can successfully target tumor site.

The high vascular permeability of renal clearable DOX@AuNPs further impacted the intratumoral distribution of drug, compared to the non-renal clearable DOX@AuNPs. At 12 h post-intravenous injection of the two DDSs into MCF-7 tumor-bearing mice, the drug distribution in tumor tissues was analysed with fluorescence microscopy imaging. The DOX carried by renal clearable AuNPs was found to extravasate from tumor vasculature and penetrated deeply into tumor microenvironment within 12 hrs (FIG. 15A). In contrast, majority of DOX delivered by large-sized NRC-DOX@AuNPs was confined within or near the blood vessels, consistent with previous observation that the large NPs showed limited tumor extravasation and penetration. Hence, for the large NRC-DOX@AuNPs, the lower drug delivery efficiency is due to the relatively lower vascular permeability than renal clearable DOX@AuNPs. Through quantitative analysis of tumor cells interacting with DOX across different distances from tumor vasculature (FIGS. 15B-D), the percentage of tumor cells interacting with DOX for the DOX@AuNPs (36-54%) was 2 to 5 times more than that of the larger NRC-DOX@AuNPs (9-19%). In addition, with increased distance-to-vessel (from 30 to 120 μm), the smaller DDS showed greater difference from larger one in the delivery efficiency to tumor cells (from 2.8 to 4.7 times, FIG. 15D). The greatly enhanced vascular permeability of renal clearable DOX@AuNPs was also found in the metastatic tumors in brain tissues in comparison with free DOX and 90 nm Doxil (FIGS. 16A-C). These findings clearly indicate that efficient intratumoral transport of DOX enabled by highly permeable DOX@AuNPs is responsible for the observed higher DOX delivery efficiency. Therefore, the renal clearable DOX@AuNPs not only carried 2-time more drug into the whole tumor but also delivered the drug into larger population of tumor cells in the tumor environment.

Because of much more efficient delivery and deeper tumor penetration of DOX with renal clearable AuNPs, therapeutic efficacy of DOX was significantly enhanced after being loaded onto the AuNPs. MCF-7 tumor-bearing mice were treated by successive injection of DOX@AuNPs, NRC-DOX@AuNPs, free DOX (equivalent DOX, 5 mg/kg body weight) and PBS, respectively, and tumor growth curves were recorded during the treatment. The normalized tumor growth curves showed that the tumor growth inhibition (TGI, compared to PBS control) of DOX@AuNPs was as high as 77.6±1.7%, whereas NRC-DOX@AuNPs and free DOX showed modest efficacies with TGI at 45.9±9.0 and 40.7±6.7%, respectively (FIG. 17A). The average tumor volume of the mice treated with DOX@AuNPs was 41.3±3.2, 37.7±2.9 and 19.9±1.5 of those of the mice treated with NRC-DOX@AuNPs, free DOX and PBS, respectively. Therefore, the survival rate of the tumor-bearing mice was significantly increased by DOX@AuNPs, as compared with free DOX, NRC-DOX@AuNPs and PBS control (FIG. 17B). As a control, renal clearable AuNPs showed no interference on the efficacy or toxicity during the study due to their high biocompatibility as well as low body accumulation. Moreover, similar tumor inhibition by DOX@AuNPs was also observed in primary murine mammary carcinoma (4T1, FIGS. 17C-D) and human triple-negative breast cancer (MDA-MB-231, FIG. 18) xenograft. In addition, since the 4T1 primary tumor showed rapid lung metastasis aggressively, the therapeutic study on 4T1 lung metastasis showed DOX@AuNPs not only effectively inhibited the lung metastasis (nodule count of 4.0±2.7 per mouse, FIGS. 17E-F), with 12.9, 15.7, 21.2 times fewer nodule counts than NRC-DOX@AuNPs, free DOX and PBS, respectively, but also decreased the nodule sizes down to 0.83±0.43 mm, significantly smaller than those treated with DOX and PBS (1.68±0.82, 1.64±0.77 and 1.95±0.90 mm, respectively, FIG. 17G). These results indicate DOX@AuNPs significantly improved antitumor efficacy than NRC-DOX@AuNPs and free DOX together with efficient renal elimination of “off-target” drug.

In addition, the antitumor efficacy of the DOX@AuNPs towards the metastatic 4T1 mammary cancer was investigated as well. After the successive treatment with DOX@AuNPs, NRC-DOX@AuNPs, DOX, Doxil and PBS, it was found that the DOX@AuNPs showed the most effective inhibition to lung metastasis of cancer cells (FIG. 19A). After DOX@AuNPs treatment, the count of tumor nodules in the lungs decreased to as low as 4.0±2.7 compared to the count of the PBS treatment (84.8±34.2), which was much lower than those after the treatments of NRC-DOX@AuNPs (51.6±20.7), DOX (62.6±30.5), and Doxil (9.0±4.6) (FIG. 19B). Moreover, the average size of the metastatic tumor nodules also decreased drastically after the treatment of DOX@AuNPs (FIG. 19C), which was 0.83±0.43 mm compared to those after the treatments of NRC-DOX@AuNPs (1.68±0.82 mm), DOX (1.64±0.77 mm), Doxil (1.06±0.52 mm) and PBS (1.95±0.90 mm). The decreased metastatic nodule count and size clearly indicated the improved efficacy versus metastatic cancers as well.

Combination of both toxicity and efficacy studies indicated that renal clearable DOX-AuNP constructs significantly enhanced the overall therapeutic index (TI) of drug. Quantitatively, the toxic dose (TD) of the DOX was increased by 1.5-fold from the administrated DOX-AuNP constructs; meanwhile, the efficacious dose (ED, defined as the dose to achieve comparable efficacy to that of free DOX treatment in FIG. 17A) was reduced by at least 6.7 times from the therapeutic study after being loaded onto the renal clearable AuNPs (FIGS. 19A-B). Hence, the renal clearable DOX-AuNP constructs showed one-order increase in TI (TD/ED) over free DOX.

With the strategic design of renal clearable DOX-AuNP constructs, the inventors enabled DOX to achieve both enhanced anticancer efficacy and minimized side effects. The renal clearable DOX-AuNP constructs have high targeting efficiency of DOX without sacrificing its tumor penetration due to the small sizes. In addition, the renal clearable DOX-AuNP constructs can also rapidly be eliminated from the normal tissues and organs through the urinary system. The differences in the targeting and clearance of DOX carried by 4-nm renal clearable AuNPs and 30-nm non-renal clearable AuNPs clearly indicates the ultrasmall renal clearable drug-AuNP constructs can overcome the multiple physiological barriers to the conventional cancer nanomedicines, for instance, the strong serum protein binding and RES recognition, and limited tumor penetration. With the rapid tumor targeting and high drug concentration in tumor environment, the overall therapeutic efficacy was significantly enhanced; with the improved renal elimination of off-target drug, the liver accumulation was reduced more than one order, the toxicity was dramatically reduced. As a result, the therapeutic index of DOX was greatly improved about 10 times. Not limited to DOX, targeting and clearance of many other potent but highly toxic chemodrugs could also be significantly improved with this new strategy, which is expected to expedite the clinical translation of cancer nanomedicines in near future.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Blanco, E., Shen, H. & Ferrari, M. Principles of nanoparticle design for overcoming biological barriers to drug delivery. Nat. Biotechnol. 33, 941 (2015).
Dai, Q. et al. Quantifying the Ligand-Coated Nanoparticle Delivery to Cancer Cells in Solid Tumors. ACS nano (2018).
Liu et al., Angew. Chem.; 52, 12572-12576, 2013.
Luk, B. T. & Zhang, L. Cell membrane-camouflaged nanoparticles for drug delivery. J. Control. Release 220, 600-607 (2015).
Shi, J., Kantoff, P. W., Wooster, R. & Farokhzad, 0. C. Cancer nanomedicine: progress, challenges and opportunities. Nat. Rev. Cancer 17, 20 (2017).
Allen, T. M. & Cullis, P. R. Drug delivery systems: entering the mainstream. Science 303, 1818-1822 (2004).
Davis, M. E. & Shin, D. M. Nanoparticle therapeutics: an emerging treatment modality for cancer. Nat. Rev. Drug Discov. 7, 771 (2008).
Iyer, A. K., Khaled, G., Fang, J. & Maeda, H. Exploiting the enhanced permeability and retention effect for tumor targeting. Drug Discovery Today 11, 812-818 (2006).
Jain, R. K. & Stylianopoulos, T. Delivering nanomedicine to solid tumors. Nature reviews Clinical Oncology 7, 653-664 (2010).
Tsoi, K. M. et al. Mechanism of hard-nanomaterial clearance by the liver. Nat. Mater. (2016).
Yu, M. & Zheng, J. Clearance pathways and tumor targeting of imaging nanoparticles. ACS nano 9, 6655-6674 (2015).
Sarin, H. Physiologic upper limits of pore size of different blood capillary types and another perspective on the dual pore theory of microvascular permeability. J. Angiogenes. Res. 2, 14 (2010).
Soo Choi, H. et al. Renal clearance of quantum dots. Nat Biotech 25, 1165-1170 (2007).
Liu, J. et al. Passive tumor targeting of renal clearable luminescent gold nanoparticles: long tumor retention and fast normal tissue clearance. J. Am. Chem. Soc. 135, 4978-4981 (2013).
Kang, H. et al. Renal Clearable Organic Nanocarriers for Bioimaging and Drug Delivery. Adv. Mater. 28, 8162-8168, doi:10.1002/adma.201601101 (2016).
Xu, J., Peng, C., Yu, M. & Zheng, J. Renal clearable noble metal nanoparticles: photoluminescence, elimination, and biomedical applications. Wiley Interdisciplinary Reviews: Nanomedicine and Nanobiotechnology (2017).
Liu, J. et al. PEGylation and Zwitterionization: Pros and Cons in the Renal Clearance and Tumor Targeting of Near-IR-Emitting Gold Nanoparticles. Angew. Chem. Int. Ed. 52, 12572-12576 (2013).
Tang, S. et al. Tailoring renal clearance and tumor targeting of ultrasmall metal nanoparticles with particle density. Angew. Chem. 128, 16273-16277 (2016).
Petros, R. A. & DeSimone, J. M. Strategies in the design of nanoparticles for therapeutic applications. Nat. Rev. Drug Discov. 9, 615, doi:10.1038/nrd2591 (2010).
Prados, J. et al. Doxorubicin-loaded nanoparticles: new advances in breast cancer therapy. Anti-Cancer Agents in Medicinal Chemistry (Formerly Current Medicinal Chemistry-Anti-Cancer Agents) 12, 1058-1070 (2012).
Tacar, O., Sriamornsak, P. & Dass, C. R. Doxorubicin: an update on anticancer molecular action, toxicity and novel drug delivery systems. J. Pharm. Pharmacol. 65, 157-170 (2013).
Shi, C. et al. A drug-specific nanocarrier design for efficient anticancer therapy. Nature Communications 6, ncomms8449 (2015).
Liao, H. et al. Antitumor efficacy of doxorubicin encapsulated within PEGylated poly (amidoamine) dendrimers. J. Appl. Polym. Sci. 131 (2014).
Lee, C. C. et al. A single dose of doxorubicin-functionalized bow-tie dendrimer cures mice bearing C-26 colon carcinomas. Proc. Natl. Acad. Sci. U.S.A. 103, 16649-16654 (2006).
Zhang, X., Chibli, H., Mielke, R. & Nadeau, J. Ultrasmall gold—doxorubicin conjugates rapidly kill apoptosis-resistant cancer cells. Bioconjugate Chem. 22, 235-243 (2010).
Kaminskas, L. M. et al. Characterisation and tumor targeting of PEGylated polylysine dendrimers bearing doxorubicin via a pH labile linker. J. Control. Release 152, 241-248 (2011).
Sun, S. et al. Dimerization of organic dyes on luminescent gold nanoparticles for ratiometric pH sensing. Angew. Chem. 128, 2467-2470 (2016).
Peng, C. et al. Targeting orthotopic gliomas with renal clearable luminescent gold nanoparticles. Nano Research 10, 1366-1376 (2017).
Chassany, O., Urien, S., Claudepierre, P., Bastian, G. & Tillement, J.-P. Comparative serum protein binding of anthracycline derivatives. Cancer Chemother. Pharmacol. 38, 571-573 (1996).
Gustafson, D. L., Rastatter, J. C., Colombo, T. & Long, M. E. Doxorubicin pharmacokinetics: macromolecule binding, metabolism, and excretion in the context of a physiologic model. J. Pharm. Sci. 91, 1488-1501 (2002).
Fundarò, A. et al. Non-stealth and stealth solid lipid nanoparticles (SLN) carrying doxorubicin: pharmacokinetics and tissue distribution after iv administration to rats. Pharmacol. Res. 42, 337-343 (2000).
Gillet, R. et al. The consequence of p53 overexpression for liver tumor development and the response of transformed murine hepatocytes to genotoxic agents. Oncogene 19, 3498 (2000).
Perrault, S. D., Walkey, C., Jennings, T., Fischer, H. C. & Chan, W. C. Mediating tumor targeting efficiency of nanoparticles through design. Nano Lett. 9, 1909-1915 (2009).
Cabral, H. et al. Accumulation of sub-100 nm polymeric micelles in poorly permeable tumors depends on size. Nat. Nanotechnol. 6, 815-823 (2011).
Wong, C. et al. Multistage nanoparticle delivery system for deep penetration into tumor tissue. Proc. Natl. Acad. Sci. U.S.A. 108, 2426-2431 (2011).
Chauhan, V. P. et al. Normalization of tumor blood vessels improves the delivery of nanomedicines in a size-dependent manner. Nat. Nanotechnol. 7, 383-388 (2012).
Xu, J. et al. Dose Dependencies and Biocompatibility of Renal Clearable Gold Nanoparticles: From Mice to Non-human Primates. Angewandte Chemie International Edition 57, 266-271 (2018).
Mahou, R. & Wandrey, C. Versatile route to synthesize heterobifunctional poly (ethylene glycol) of variable functionality for subsequent pegylation. Polymers 4, 561-589 (2012).
Liu, J. et al. Luminescent Gold Nanoparticles with Size-Independent Emission. Angew. Chem. 128, 9040-9044 (2016)Tran, T.-H. et al. Long circulating self-assembled nanoparticles from cholesterol-containing brush-like block copolymers for improved drug delivery to tumors. Biomacromolecules 15, 4363-4375 (2014).
Alhareth, K., Vauthier, C., Gueutin, C., Ponchel, G. & Moussa, F. HPLC quantification of doxorubicin in plasma and tissues of rats treated with doxorubicin loaded poly (alkylcyanoacrylate) nanoparticles. J. Chromatogr. B 887, 128-132 (2012).
Gabizon, A. A. Selective tumor localization and improved therapeutic index of anthracyclines encapsulated in long-circulating liposomes. Cancer Res. 52, 891-896 (1992).
Laginha, K. M., Verwoert, S., Charrois, G. J. & Allen, T. M. Determination of doxorubicin levels in whole tumor and tumor nuclei in murine breast cancer tumors. Clin. Cancer. Res. 11, 6944-6949 (2005).
Du, B. et al. Glomerular barrier behaves as an atomically precise bandpass filter in a sub-nanometre regime. Nat. Nanotechnol. 12, nnano. 2017.2170 (2017).
MacKay, J. A. et al. Self-assembling chimeric polypeptide—doxorubicin conjugate nanoparticles that abolish tumors after a single injection. Nat. Mater. 8, 993 (2009).
Qian, H., Zhu, Y. & Jin, R. Atomically precise gold nanocrystal molecules with surface plasmon resonance. Proc. Natl. Acad. Sci. U.S.A. 109, 696-700 (2012).
Qian, H., Zhu, M., Gayathri, C., Gil, R. R. & Jin, R. Chirality in gold nanoclusters probed by NMR spectroscopy. ACS nano 5, 8935-8942 (2011).

1. A composition comprising a noble metal nanoparticle comprising at least a first drug, wherein the surface is derivatized with polyethylene glycol and benzyl mercaptan or derivative thereof, and wherein said drug is non-covalently associated with the benzyl mercaptan or derivative thereof.

2. The composition of claim 1, wherein the noble metal is gold.

3. The composition of claim 1, wherein the nanoparticle is about 0.5 nm to 10 nm in diameter.

4-5. (canceled)

6. The composition of claim 1, wherein the nanoparticle is derivatized with a benzyl mercaptan methoxy derivative, mercaptobenzoic acid, aminothiophenol, methylbenzenethiol, thiolated pyrene, thiolated nucleobases or other aromatic thiol.

7-8. (canceled)

9. The composition of claim 1, wherein the polyethylene glycol and benzyl mercaptan or derivative thereof shield loaded drugs and stabilize the nanoparticles.

10. (canceled)

11. The composition of claim 9, wherein the nanoparticle is renally clearable.

12-15. (canceled)

16. The composition of claim 1, wherein the drug is a nucleic acid.

17. The composition of claim 16, wherein the nucleic acid is an siRNA.

18. The composition of claim 1, wherein drug is a small molecule, such as doxorubicin, daunorubicin, epirubicin, idarubicin, cisplatin, oxaliplatin, carboplatin, or nedaplatin.

19-23. (canceled)

24. The composition of claim 18, wherein the small molecule is an imaging agent.

25. The composition of claim 24, wherein the imaging agent is a near-infrared imaging agent, such as indocyanine green and methylene blue.

26. The composition of claim 24, wherein the imaging agent is a gadolinium complex.

27. (canceled)

28. The composition of claim 1, wherein the nanoparticle further comprises an imaging probe or radiotracer.

29. The composition of claim 1, wherein the noble metal consists of, comprises, or consists essentially of silver, copper, platinum, or carbon.

30-31. (canceled)

32. The composition of claim 1, wherein said non-covalent bond involves pi-pi stacking or electrostatic interactions.

33. (canceled)

34. The composition of claim 1, wherein the nanoparticle achieves rapid and high drug accumulation in tumor tissues as compared to a nanoparticle lacking benzyl mercaptan derivatization.

35. (canceled)

36. The composition of claim 1, wherein the nanoparticle reaches a penetration of about 40% or more of tumor cells within a distance of about 100 μm of a blood vessel in the tumor environment.

37-39. (canceled)

40. The composition of claim 1, wherein the nanoparticle targets solid tumors, including primary and metastatic ones.

41. The composition of claim 1, wherein the nanoparticle penetrates normal tissues via endothelial junctions.

42. The composition of claim 1, wherein the nanoparticle is smaller than interendothelial junctions and permeable into skeletal muscle (gastrocnemius) of a subject show reduced retention in normal organs and tissues, such as skeletal muscle, heart, lung, and liver as compared to a nanoparticle lacking benzyl mercaptan derivatization.

43. A method of treating a subject having a hyperproliferative disease comprising administering to the subject a composition comprising of claim 1.

44-46. (canceled)

47. The method of claim 43, wherein the subject is a human subject.

48-52. (canceled)
